(19) United States
(12) Patent Application Publication
(10) Pub. No.: US 2023/0226013 A1
(43) Pub. Date: Jul. 20, 2023

(54) COMPOSITION AND METHOD FOR TREATING CANCER

(71) Applicant: Carmel-Haifa University Economic Corp Ltd., Haifa (IL)

(72) Inventors: Fuad FARES, Hourfish Village (IL); Lital SHARVIT, Kibbuz Meggido (IL); Solomon WASSER, Nesher (IL)

(21) Appl. No.: 17/756,899

(22) PCT Filed: Dec. 3, 2020
(86) PCT No.: PCT/IL2020/051245

§ 371 Date: Jun. 3, 2022

Related U.S. Application Data
(60) Provisional application No. 63/070,931, filed on Aug. 27, 2020.
(60) Provisional application No. 62/943,275, filed on Dec. 4, 2019.

(51) Int. Cl. A61K 31/35; A61K 36/07; A61K 45/06

(52) U.S. Cl. CPC A61K 31/35; A61K 36/07; A61K 45/06; A61K 2236/39

(57) ABSTRACT

A fractionation method, pure active compounds and derivatives thereof, compositions including the same and methods of treating cancer including administering such compounds, derivatives, compositions or any combination thereof. A fractionation method of a Cyathus striatus CBS 126585 for obtaining at least one of the pure active ingredients Striatal C, Striatal C′ or Striatal D, wherein the fractionation method includes: providing an octadecyl silica gel column; and performing a series of RP-18 preparative chromatography steps using the acetadecyl silica gel column.

### FIELD OF THE INVENTION

This invention is directed to a fractionation method of a Cyathus striatus CBS 126585 extract for obtaining pure active ingredients. Further, the invention is directed to a process for synthesizing the pure active ingredients. In addition, the invention is directed to derivatives of the pure active ingredients and to processes for synthesizing the same. The invention is further directed to a composition comprising the pure active ingredients and/or derivatives thereof, as well as to a method of treating cancer comprising administering the same.

### BACKGROUND OF THE INVENTION

Natural products are known to be a significant source of drugs. Many conventional therapies have been developed from nature-derived materials. Their dominant role in cancer chemotherapeutics is clear, where about 75% of the known anticancer compounds are either natural products or natural product-derived. It is estimated that approximately 25% of the drugs prescribed worldwide at present are derived from plants and that 60% of antitumor/anti-infectious drugs already on the market or under clinical investigations are of natural origin.

PCT International Patent Application Publication No. WO 2011/151831 discloses a Cyathus striatus CBS 126585 extract; however, as detailed therein, the Cyathus striatus CBS 126585 extract includes many active ingredients, each of which may act using a different pathway. It was hypothesized therein that the combination of the active ingredients in the extract is beneficial in treating cancer.

Pancreatic ductal adenocarcinoma (PDAC) is one of the most fatal solid malignancies. It is the thirteenth most common cancer worldwide and one of the leading causes of cancer death with more than 331,000 deaths per year, worldwide. In the United States and Israel, pancreatic cancer is the fourth leading cause of cancer related mortalities. In 2010, about 36,800 patients died from pancreatic cancer in the US alone.

PDAC is known to have a very poor prognosis mainly because of the insensitivity thereof to most standard therapies including, chemotherapy, radiotherapy, and immunotherapy. Therefore, surgical resection offers, at the moment, the only potential prospect for a cure. For all stages combined, the 1- and 5-year relative survival rates of PDAC are 24% and 5%, respectively. Even for those people diagnosed with the local form of the disease, the 5-year survival is merely 20%. These unfortunate statistics reflect the advanced stage at which most patients with pancreatic cancer are diagnosed and the paucity of effective chemotherapeutic regimens for the advanced disease.

### SUMMARY OF THE INVENTION

Some embodiments of the invention are directed to a fractionation method of a Cyathus striatus CBS 126585 for obtaining at least one of the pure active ingredients Striatal C, Striatal C′ and Striatal D

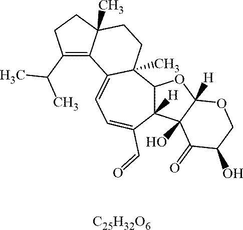

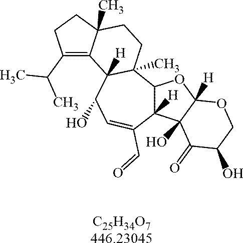

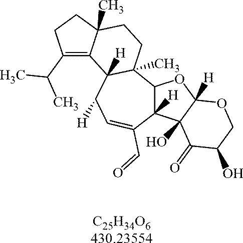

wherein the fractionation method includes providing an octadecyl silica gel column; and performing a series of RP-18 preparative chromatography steps using the acetadecyl silica gel column, wherein the elution solvents are double distilled water (DDW, solvent A) and acetonitrile (ACN, solvent B), and wherein the first step includes eluting with 100% solvent A and 0% solvent B, and wherein the amount of solvent A was decreased by 5% and the amount of solvent B was increased by 5% in each 10 minute interval, until reaching a solvent comprising 100% solvent B, and wherein the elution solvents were passed through the octadecyl silica gel column at a predefined flow rate.

According to some embodiments, the fractionation method further comprises passing 100% solvent B through the octadecyl silica gel column for an additional 20-40 minutes.

According to some embodiments, the predefined flow rate is 3-7 ml/min.

Further embodiments of the invention are directed to compounds according to the following formula:

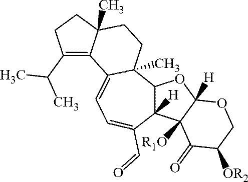

wherein R₁ and R₂ may be the same or different and are selected from H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde.

Further embodiments of the invention are directed to compounds according to the following formulae:

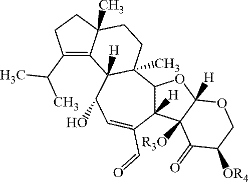

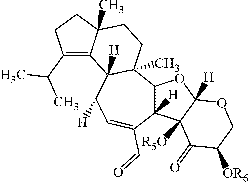

wherein R₃, and R₄ may be the same or different and are selected from H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde; and
wherein R₅, and R₆ may be the same or different and are selected from H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde.

Further embodiments of the invention are directed to compounds according one of the following formulae:

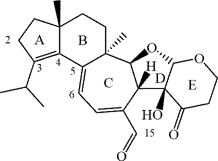

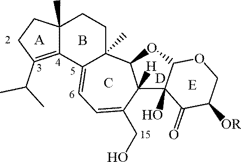

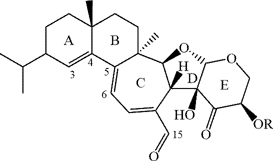

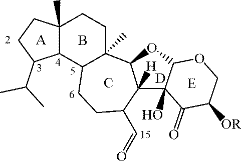

Some embodiments of the invention are directed to a process for synthesizing Striatal A or Striatal C, wherein the process includes the following steps:

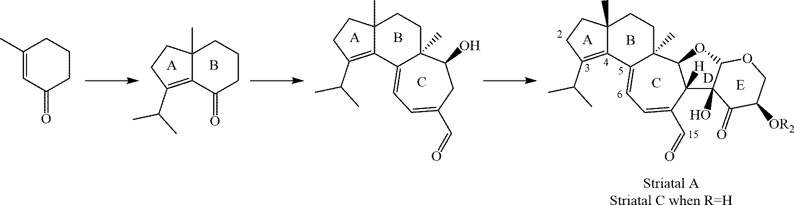

wherein R₂ is an acetyl group.

Further embodiments of the invention are directed to a composition comprising Striatal A, Striatal C, Striatal C′, Striatal D, at least one derivative of Striatal A, Striatal C, Striatal C′, Striatal D, or any combination thereof.

According to some embodiments, the composition further comprises a carrier, diluent, solvent, buffer, fragrance, colorants, taste enhancers, polyethylene glycol (PEG), albumin, nanoparticles, or any combination thereof.

According to some embodiments, the composition further comprises or is administered together with an additional active ingredient. According to some embodiments, the additional active ingredient is an anti-cancer drug.

According to some embodiments, the composition further comprises or is administered together with a food supplement, a probiotic, a prebiotic, a nutraceutical, a beverage product, a cosmetic product, any combination thereof.

According to some embodiments, the composition further comprises or is administered together with the biomass, extract, or any combination thereof, of any other medicinal mushrooms.

According to some embodiments, the medicinal mushroom is selected from the Lentinus edodes, Coprinus and Tremella species.

According to some embodiments, the composition further comprises or is administered together with a Cyathus striatus CBS 126585 extract.

Further embodiments of the invention are directed to a method of treating cancer comprising administering Striatal A, Striatal C, Striatal C′, Striatal D, at least one derivative of Striatal A, Striatal C, Striatal C′, Striatal D, or any combination thereof, or a composition comprising the same, to a person in need of such treatment.

According to some embodiments, the cancer is epithelial cell cancer.

According to some embodiments, the cancer is pancreatic cancer, breast cancer, colon and rectal cancer, head and neck cancer, chronic myelogenous leukemia (CML),prostate cancer, ovary cancer or uterus cancer. According to some embodiments, the pancreatic cancer is pancreatic ductal adenocarcinoma (PDAC). According to some embodiments, the cancer is glioblastoma, melanoma, myeloma, endometrium cancer, adrenal cancer or thyroid cancer.

### BRIEF DESCRIPTION OF THE DRAWINGS

Some embodiments of the invention will be understood and appreciated more fully from the following detailed description in conjunction with the figures, which are not to scale, in which like reference numerals indicate corresponding, analogous or similar elements, and in which:

FIG. 1 presents an HPLC chromatogram of the Cyathus striatus CBS extract (2000 ppm, Detector UV/VIS);

FIG. 2 presents an HPLC chromatogram of purified Striatal C (2000 ppm, Detector UV/VIS);

FIG. 3 presents the electrospray ionization mass spectrometer (ESI scan) of purified Striatal C and Striatal C′;

FIG. 4 presents a comparison between the effect of Striatal C and the Cyathus striatus CBS 126585 extract on the viability of HPAF-II and PL45 human pancreatic cancer cells;

FIG. 5 presents a comparison between the effect of Striatal C and the Cyathus striatus CBS 126585 extract on the lactate dehydrogenase (LDH) leakage in HPAF-II and PL45 human pancreatic cancer cells;

FIG. 6 presents a comparison between the effect of Striatal C and the Cyathus striatus CBS 126585 extract on the induction of apoptosis in HPAF-II and PL45 human pancreatic cancer cells;

FIG. 7 presents analytical HPLC UV chromatogram of crude sample “C.S. Extraction 19.12.19” ;

FIG. 8 presents the typical Preparative HPLC chromatogram of sample “C.S. Extraction 19.12.19” ;

FIG. 9 presents analytical HPLC UV/MS chromatogram of isolated compound;

FIG. 10 presents HPLC-MS ESI⁺ spectra of chromatographic peaks;

FIG. 11 presents the chemical structure of the isolated compound;

FIG. 12 presents ¹H-NMR spectrum of the sample “300582-42”; and

FIG. 13 presents ¹³C-NMR (H decoupled) spectrum of the “300582-42”.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Throughout this description, the terms “about”, “approximately”, and the like, are intended to cover ±10% of the specifically disclosed value. Further, the term “treating cancer”, as used herein refers to the inhibition of the growth of cancer cells. Such treatment may lead to the regression of tumor growth, including the decrease in size or complete regression of the tumor. Further, the term “treating cancer” as used herein, may also include alleviating or curing the disseminated tumors, namely, of metastases, as well as postponing the progression of the cancer by any means and for any length of time.

Embodiments of the invention are directed to a fractionation method of a Cyathus striatus CBS 126585 extract for obtaining the pure active ingredients.

In some embodiments, the purity of the active ingredients is between 95% to 100%. In other embodiments, the purity of the active ingredients is between 96% to 99%. In other embodiments, the purity of the active ingredients is between 97% to 99%. In other embodiments, the purity of the active ingredients is between 98% to 99%.

The pure active ingredients are referred to herein as Striatal C, Striatal C′ and Striatal D and are represented by the following chemical formulae:

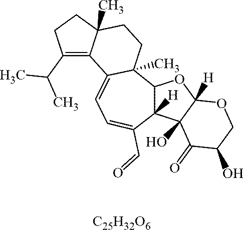

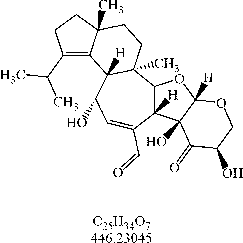

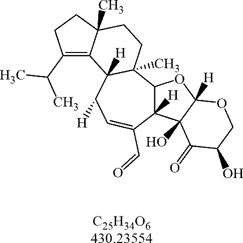

The fractionation method of the invention includes:
providing an octadecyl silica gel column; and
performing a series of RP-18 preparative chromatography steps using the acetadecyl silica gel column,
wherein the elution solvents were double distilled water (DDW, solvent A) and acetonitrile (ACN, solvent B), wherein the first step including eluting with 100% solvent A and 0% solvent B, and wherein the amount of solvent A was decreased by 5% and the amount of solvent B was increased by 5% in each 10 minute interval, until reaching a solvent comprising 100% solvent B.

According to some embodiments, the fractionation method further comprises passing 100% solvent B through the column for an additional 20-40 minutes. According to some embodiments, the fractionation method further comprises passing 100% solvent B through the column for an additional 30 minutes. According to some embodiments, the fractionation method further comprises passing 100% solvent B through the column for an additional 30-60 minutes.

According to some embodiments, the flow rate in the fractionation method is between about 3-7 ml/min. According to some embodiments, the flow rate in the fractionation method is about 5 ml/min.

Further embodiments of the invention are directed to derivatives of Striatal C. According to some embodiments, the derivatives of Striatal C are referred to herein as “Striatal A”, having the chemical formula:

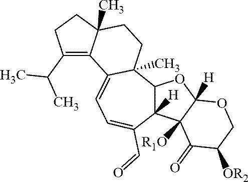

According to some embodiments, R₁ and R₂ may be the same or different and are selected from H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde.

Further embodiments of the invention are directed to derivatives of Striatal C′ and Striatal D. According to some embodiments, the derivatives of Striatal C′ and Striatal D are referred to herein as Striatal A′ and Striatal B respectfully, having the chemical formula:

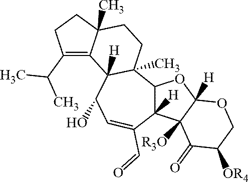

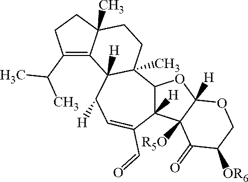

According to some embodiments, R₃, and R₄ may be the same or different and are selected from H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde.

According to some embodiments, R₅, and R₆ may be the same or different and are selected from H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde.

According to further embodiments, the derivatives of Striatal C, Striatal C′ and/or Striatal D are according to the formulae detailed in Scheme 1 below, and are related to herein as “Derivative 1”, “Derivative 2”, “Derivative 3” and “Derivative 4”:

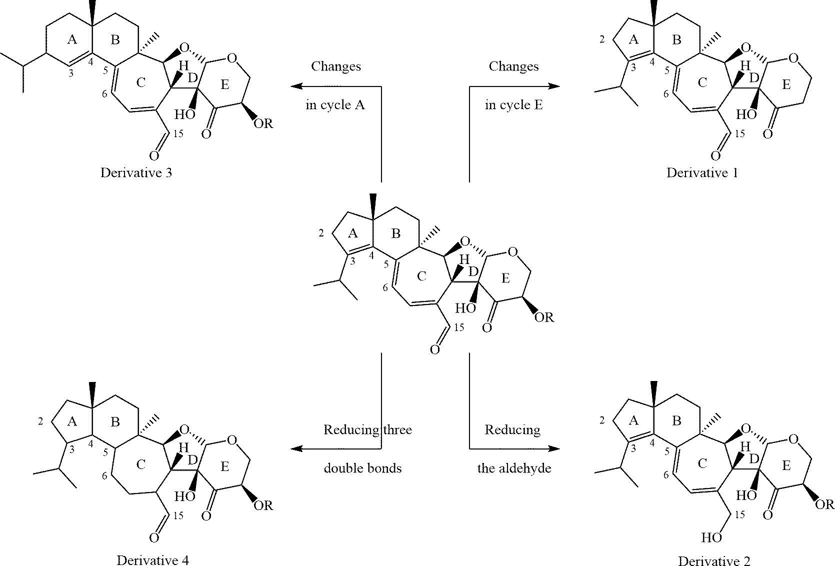

Further embodiments of the invention are directed to a process for chemically synthesizing Striatal A, wherein the process comprises:

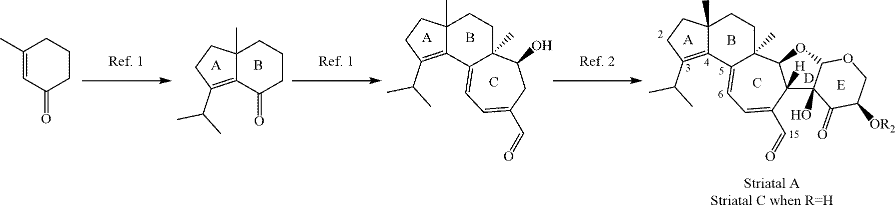

wherein, if R₂ is H, the Striatal is Striatal C. According to some embodiments, R₂ is an acetyl group.

Further embodiments of the invention are directed to a composition comprising Striatal A, Striatal C, Striatal C′, Striatal D, at least one derivative of Striatal A, Striatal C, Striatal C′, Striatal D, or any combination thereof.

The composition may further comprise any known additive, such as a carrier, diluent, solvent, buffer, fragrance, colorants, taste enhancers, polyethylene glycol (PEG), albumin, nanoparticles, or any combination thereof.

According to some embodiments, the composition further comprises any additional active ingredient, including any type of known anti-cancer drug. The composition may further comprise a food supplement, a probiotic, a prebiotic, a nutraceutical, a beverage product, a cosmetic product, any combination thereof or the like.

According to some embodiments, the composition may comprise the biomass, extract, or any combination thereof, of any other medicinal mushrooms such as, but not limited to, Lentinus edodes, Coprinus and Tremella species. According to some embodiments, Striatal A, Striatal C, Striatal C′, Striatal D, at least one derivative of Striatal A, Striatal C, Striatal C′, Striatal D, or any combination thereof, may be administered together with a Cyathus striatus CBS 126585 extract.

Any two active ingredients may be administered in a single composition or in separate compositions administered at the same time, sequentially, at certain, predetermined time intervals, etc. Further, if more than one composition is administered, each composition may be administered in a different form and by different means.

The composition of the invention may be in any solid, liquid or gas form. Further, the composition of the invention and may be administered by any appropriate means, including orally, intravenously, topically, subcutaneously, intraarticularly, intramuscularly, by inhalation, intranasally, intrathecally, intraperitoneally, intradermally, transdermally or enterally.

Further embodiments of the invention are directed to a method of treating cancer comprising administering Striatal A, Striatal C, Striatal C′, Striatal D, at least one derivative of Striatal A, Striatal C, Striatal C′, Striatal D, or any combination thereof, to a person in need of such treatment. The method may include the direct administration of any of the active ingredients or the administration of a composition comprising any of the active ingredients.

According to some embodiments, the cancer is epithelial cell cancer, carcinoma, sarcoma, leukemia, lymphoma, melanoma or germinoma. According to other embodiments, the carcinoma is adenocarcinoma, squamous cell carcinoma, adenosquamous cell carcinoma, anaplastic carcinoma, large cell carcinoma or small cell carcinoma.

According to some embodiments, the cancer is pancreatic cancer, breast cancer, ovarian cancer, uterine cancer, chronic myelogenous leukemia (CML), prostate cancer, bladder cancer, colon and rectal cancer, head and neck cancer, endometrial cancer, kidney cancer, liver cancer, lung cancer, non-Hodgkin lymphoma or thyroid cancer. According to some embodiments, the cancer is pancreatic cancer. According to some embodiments, the pancreatic cancer is pancreatic ductal adenocarcinoma (PDAC). According to some embodiments, the cancer is glioblastoma, melanoma, myeloma, endometrium cancer, adrenal cancer or thyroid cancer.

Unless explicitly stated, the method embodiments described herein are not constrained to a particular order or sequence. Additionally, some of the described method embodiments or elements thereof can occur or be performed simultaneously, at the same point in time, or concurrently.

Various embodiments have been presented. Each of these embodiments may, of course, include features from other embodiments presented, and some embodiments not specifically described may include various features described herein.

### EXAMPLES

As can be well appreciated by a person of skill in the art, HPLC peaks as presented in the examples below, may vary between different HPLC methods and conditions. It is to be understood that the described invention is not limited by the peaks values or any other methods described in the examples below.

### Preparation of the Crud Extract

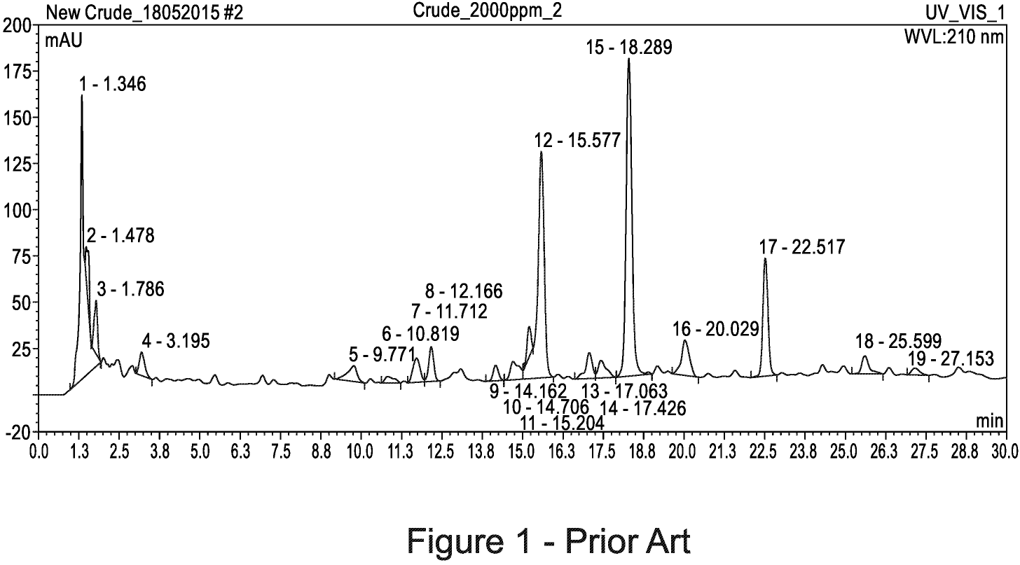

A Cyathus striatus CBS 126585 extract was prepared by harvesting and extracting Cyathus striatus CBS using ethyl acetate as the extraction solvent. After evaporation of the ethyl acetate, a crude extract was obtained. Injection of the crude extract to an HPLC combined to a diode array detector, provided the chromatogram in FIG. 1 (Prior Art).

### Example 1 - The Isolation and Purification of Striatal C

Striatal C (presented by the 16 min. peak shown in FIG. 1) was purified from 0.5 gr of the crude Cyathus striatus CBS extract by a series of RP-18 preparative chromatography using an octadecyl silica gel column, to obtain the 5 mg of purified Striatal C.

The solvents used to eluate the extract fractions were (A)-DDW and (B)- acetonitrile (ACN). The chromatography started with 0% of solvent B then it increased by 5% each 10 min. to 100% B and kept at 100% B for another 30 min. flow rate was 5 ml/min.

### HPLC Analysis

Samples of the extract were analyzed by injecting 20 µL of the chromatographed fraction solutions (2 mg mL⁻¹ in methanol) into a UHPLC connected to a photodiode array detector (Dionex Ultimate 3000), with a reverse-phase column (Phenomenex RP-18, 150 _ 4.0 mm, 3 µm). The mobile phase consisted of (A) DDW with 0.1% formic acid and (B) acetonitrile containing 1% formic acid, run at a gradient starting from 40% B then increased to 90% B for 22 min, and kept at 90% B for another 8 min, at a flow rate of 1 mL min⁻¹.

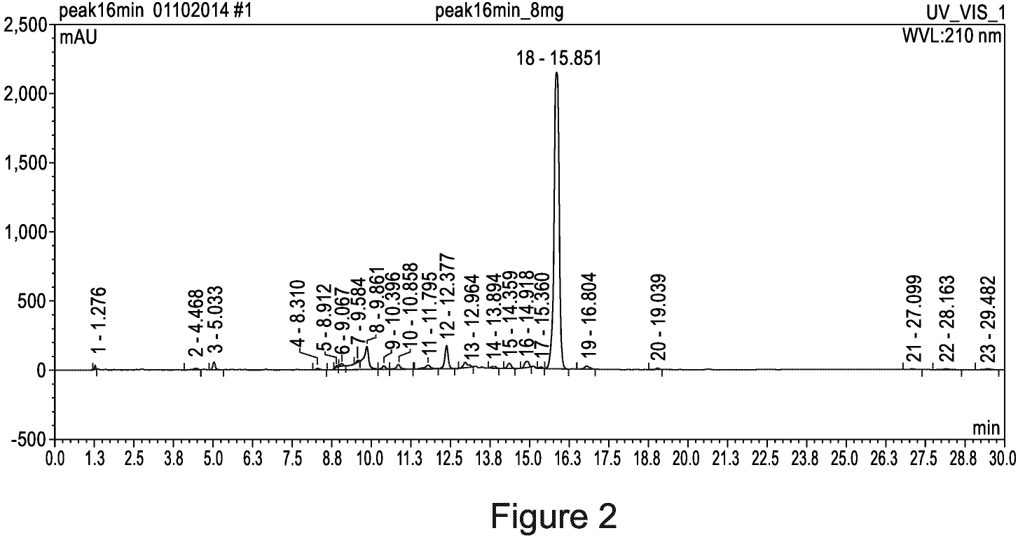

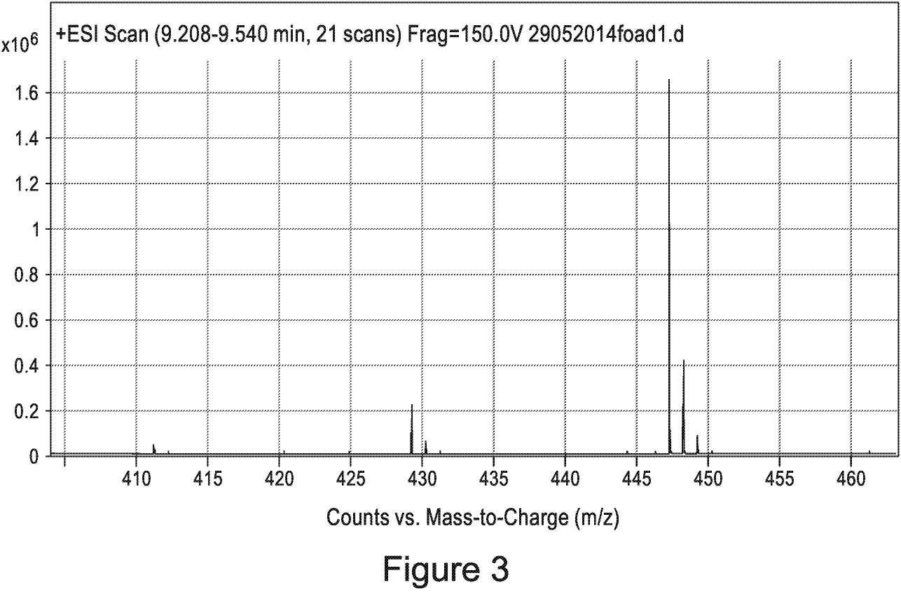

The purified Striatal C had the HPLC chromatogram shown in FIG. 2, and the electrospray ionization mass spectrometer scan (ESI scan) of Striatal C is shown in FIG. 3. It is noted that Straital C has a retention time (RT) of 16 min, while Striatal C′ has an RT of 13 minutes. The highest peak in FIG. 3 is for the Striatal C, i.e., RT16, while the lower peak, i.e., the second highest peak, is for Striatal C′, i.e., RT13.

According to some embodiments, Striatal C is more effective as an active ingredient than Striatal C′. According to some embodiments, Striatal C is the most effective active ingredient in the extract.

Particularly, UV/VIS of the pure compound with the RT of 13 min, i.e., Straital C′, revealed that it has one maximum at λmax of 193. FTIR, H1 and C-13 NMR, as well as mass spectra analysis at high resolution of the RT 13 min peak further shows that its empirical formula is of C₂₅H₃₄O₇ which, under the HPLC conditions, is in equilibrium with the compound having the RT 16 min peak in the HPLC diagram, wherein the empirical formula of the RT 16 min peak compound is C₂₅H₃₂O₆, i.e., Striatal C (elimination of one water molecule in comparison to the compound of RT 13 min). Further, the RT 16 min compound (Striatal C) provides two maxima UV/VIS at λmax 194 and 234 (elimination of water, H₂O, occurred and formation of conjugated double bond).

The peak at RT 16 (Striatal C, C₂₅H₃₂O₆) has ten degrees of unsaturation and from the NMR it is clear that this molecule contains two carbonyl groups, one simple keto group and the second, an aldehyde. The reaction of Striatal C and Striatal C′ with diazo compound (Dinitro phenyl hydrazine) to form a stable imine derivative shows that both Striatal C and Striatal C′ indeed contain an aldehyde group (see the formulae for Striatal C and Striatal C′ above)

### Pharmaceutical Properties of Striatal C

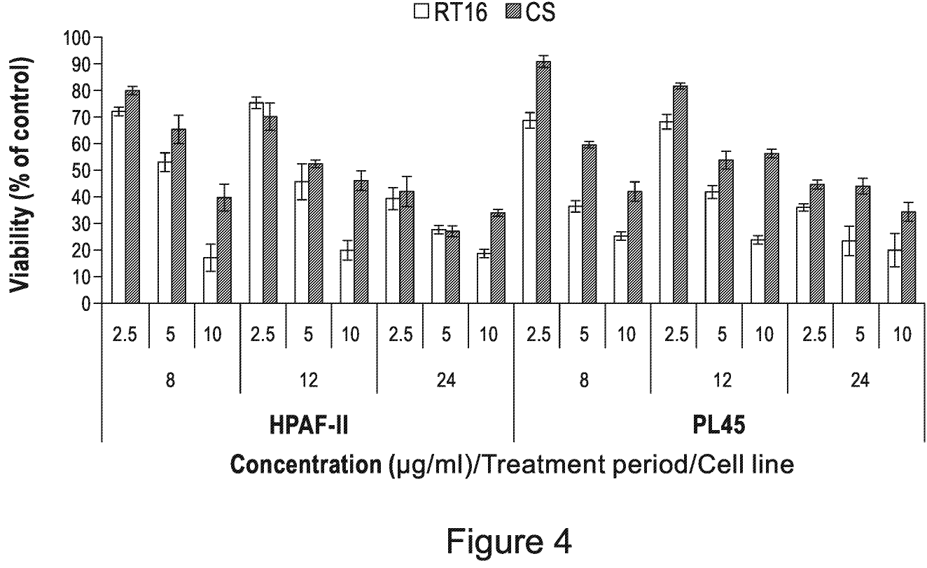

Reference is now made to FIG. 4, presenting the effect of Striatal C on the viability of HPAF-II and PL45 human pancreatic cancer cells, in comparison to the effect of the Cyathus striatus CBS 126585 extract on the same cells, wherein the measurements of the effects are based on an XTT assay. Particularly, cell proliferation was determined using the sodium 3′-[1-(phenylaminocarbonyl)-3,4-tetrazolium]-bis (4-methoxy-6-nitro) benzene sulfonic acid hydrate (XTT) assay, according to the manufacturer’s instructions (Biological Industries, Beit Haemek, Israel).

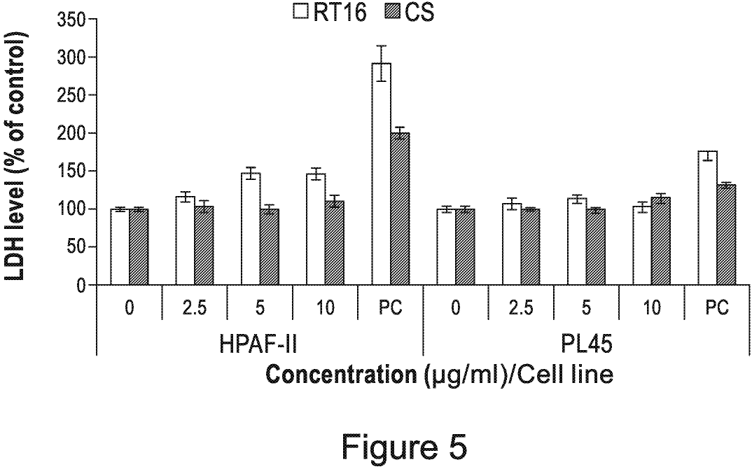

Reference is now made to FIG. 5, presenting the effect of Striatal C on lactate dehydrogenase (LDH) leakage in HPAF-II and PL45 human pancreatic cancer cells, in comparison to the effect of the Cyathus striatus CBS 126585 extract on the same cells, based on the LDH cytotoxicity assay. The LDH test was used to evaluate cytotoxicity of the extract on the cell lines. LDH is rapidly released into the medium when the plasma membrane is damaged. The integrity of the plasma membrane following treatment was determined by measuring LDH activity, released into the culture medium. the levels of LDH released into the cell culture supernatant was detected by LDH Cytotoxicity Detection Kit ^{PLUS} (Roche, Mannheim, Germany) following the manufacturer’s instructions.

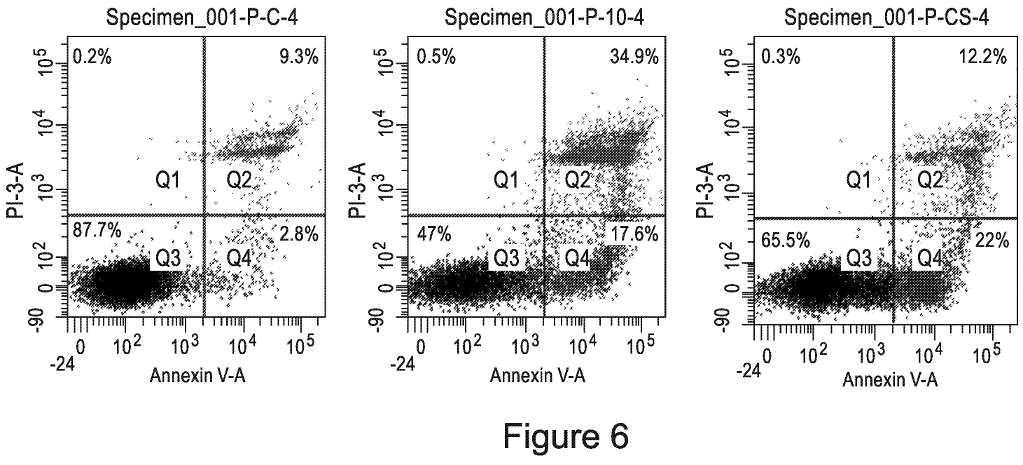

Reference is now made to FIG. 6, presenting the effect of Striatal C on the induction of apoptosis in HPAF-II and PL45 human pancreatic cancer cells, in comparison to the effect of the Cyathus striatus CBS 126585 extract on the same cells, based on Annexin-V/PI assay. Particularly, Cell death was analyzed by staining the cells with FITC-labeled Annexin V and propedium iodide (PI) using an Annexin V-FITC apoptosis detection kit, (MBL, Nagoya, Japan), according to the manufacturer’s instructions.

The results show that the treatment of human pancreatic cancer cells with low doses of Striatal C exhibited a profound decrease in cell viability and an induction of apoptosis following a period of 4 hours of exposure to the Striatal C fraction. The control used had 0 µg/ml and the most effective concentration was 10 µg/ml.

### Example 2 - The Isolation and Identification of Active Compound Striatal C′

The crude extract was received for isolation feasibility of the active compound. The marker of active compound, provided by CanCurX, was used for development of analytical HPLC and Prep HPLC methods in order to isolate and identify chemical structure of unknown compound. At second step: two grams of crude extract were separated by Prep HPLC in order to evaluate mass yield (concentration) of active compound in original crude extract and elucidate chemical structure by various analytical spectroscopic methods.

### Summary

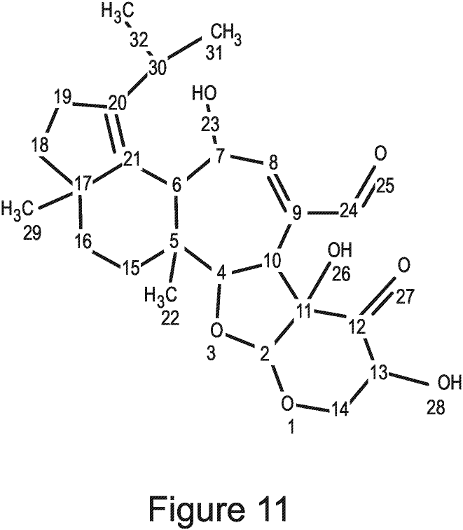

1. Interpretation of NMR and MS data led us to propose follow structure of the isolated compound (FIG. 11).

2. The isolated compound was stable and pure (purity > 95% by HPLC) at solid state conditions at -20° C.

3. Separation of 2000 mg crude material “C.S. Extraction 19.12.19” by Preparative HPLC provide 204 mg of the isolated compound.

4. During NMR experiments observed partially isomerization of isolated compound. The dissolving in dichloromethane at 20 mg/ml concentration leads to steady state between major and minor isomers with ratio ~75:25. HPLC analyses of NMR solution show only one peak of isolated compound.

Analytical HPLC conditions (Monitoring of Preparative separation and Scale up of Preparative separation):
Pumping system: Agilent model 1200
Detector: Agilent model 1200
Column: Phenomenex Gemini C-18,110A, 250*4.6 mm*10micron
Column Temperature: 25° C.
Sample temperature: 4° C. Detector :
$${ƛ}_{220 n m}$$
Mobile phase: A (Acetonitrile) and B (DI water) according to gradient program:

| Time | %A (Acetonitrile) | %B (DI water) |
|---|---|---|
| 0 | 50 | 50 |
| 22 | 50 | 50 |
| 30 | 100 | 0 |
| 35 | 100 | 0 |
| 37 | 50 | 50 |

Flow: 1.5 ml/min.

Preparative HPLC conditions:
Pumping system: Waters 579 model DSC
Detector: Knauer 2500 UV/VIS
Column: Phenomenex Gemini C-18,110A, 250*21.2 mm*10 micron
Mobile phase: A (Acetonitrile) and B (DI water) according to gradient program:

| Time | %A (Acetonitrile) | %B (DI water) |
|---|---|---|
| 0 | 50 | 50 |
| 30 | 50 | 50 |
| 32 | 100 | 0 |
| 50 | 100 | 0 |

Flow: 31 ml/min.

Column Temperature: RT
Detector:
$${ƛ}_{220 n m}$$
Injection: 2 ml (sample concentration is 50 mg/ml in acetonitrile).

### LC-MS:

Pumping system: Agilent-Technologies model 1290 series
UV/Vis Detector: Agilent model 1290 series
MS Detector: Agilent-Technologies model 6550 iFunnel Q-TOF LC/MS, ESI⁺
Column: Phenomenex Gemini C-18, 110A, 250*4.6 mm*10 micron
Mobile phase: A (Acetonitrile) and B (DI water) according to gradient program:

| Time | %A (Acetonitrile) | %B (DI water) |
|---|---|---|
| 0 | 50 | 50 |
| 22 | 50 | 50 |
| 23 | 90 | 10 |
| 33 | 90 | 10 |
| 34 | 50 | 50 |

Flow: 1.5 ml/ml.

Column temp: 25° C.

### NMR:

¹H and ¹³C NMR spectra were acquired on a Bruker AVANCE III (500 MHz) spectrometer at 296-301 K, and CD₂Cl₂ as a solvent.

¹H-NMR (500 MHz) chemical shifts were calculated using the solvent resonance as internal standard (¹H:5.32 ppm for CD₂Cl₂ signal).

¹³C-NMR (125.76 MHz) chemical shifts were calculated using the solvent resonance as internal standard (¹³C{¹H}: 54.00 ppm for CD₂Cl₂ signal).

### Results

Two grams of crude extract were separated by Prep HPLC in order to evaluate mass yield (concentration) of active compound in original crude extract and elucidate chemical structure of isolated compound by spectroscopic methods.

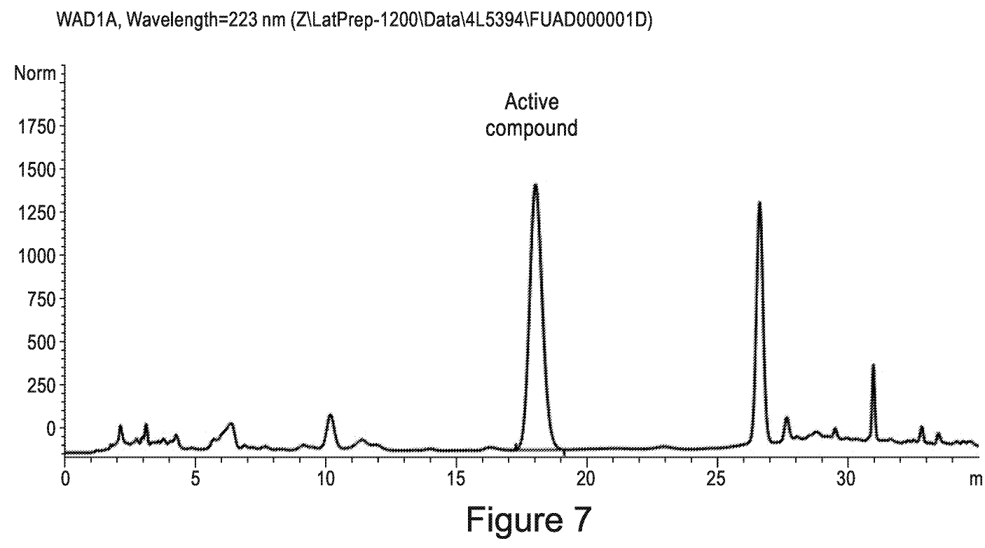

Preparative HPLC was used for the isolation of the Active compound. For this purpose, a Preparative HPLC method was developed on analytical HPLC column Phenomenex Gemini C-18 (FIG. 7) Rt (Active compound) = 17.9 min.

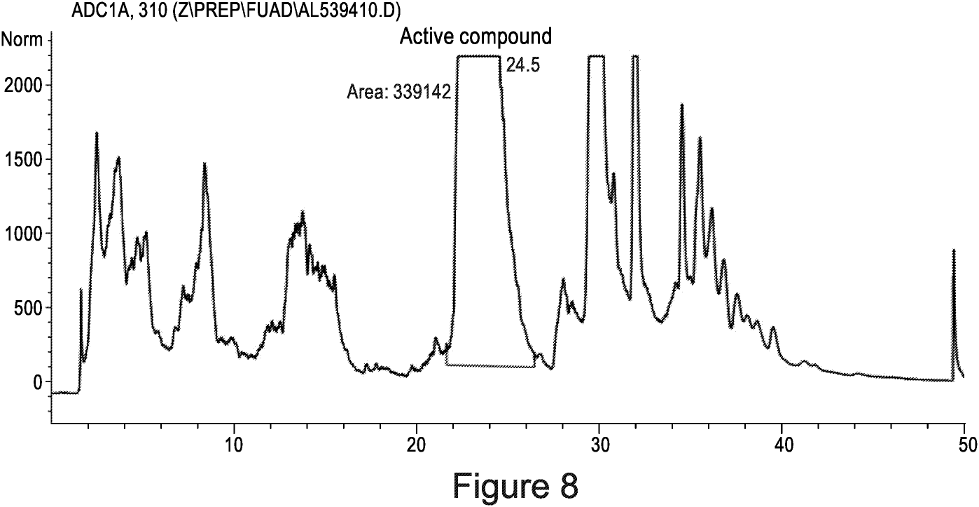

The Preparative HPLC (FIG. 8) fractions at Rt 23-26 min was collected and monitored by HPLC. Typical Rt (Active compound) = 23-26 min.

### HPLC/MS Analysis

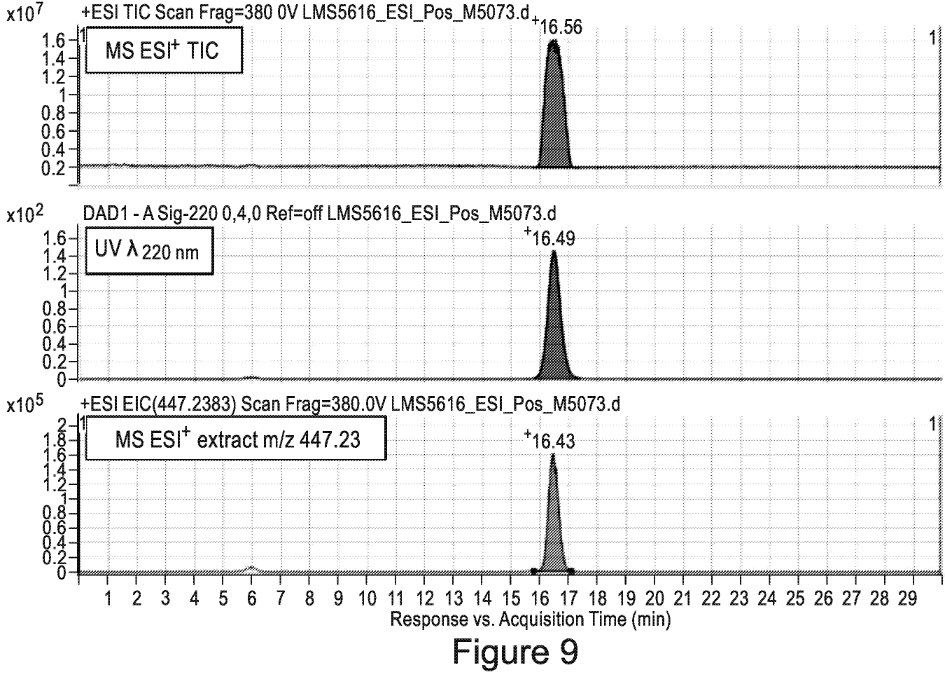

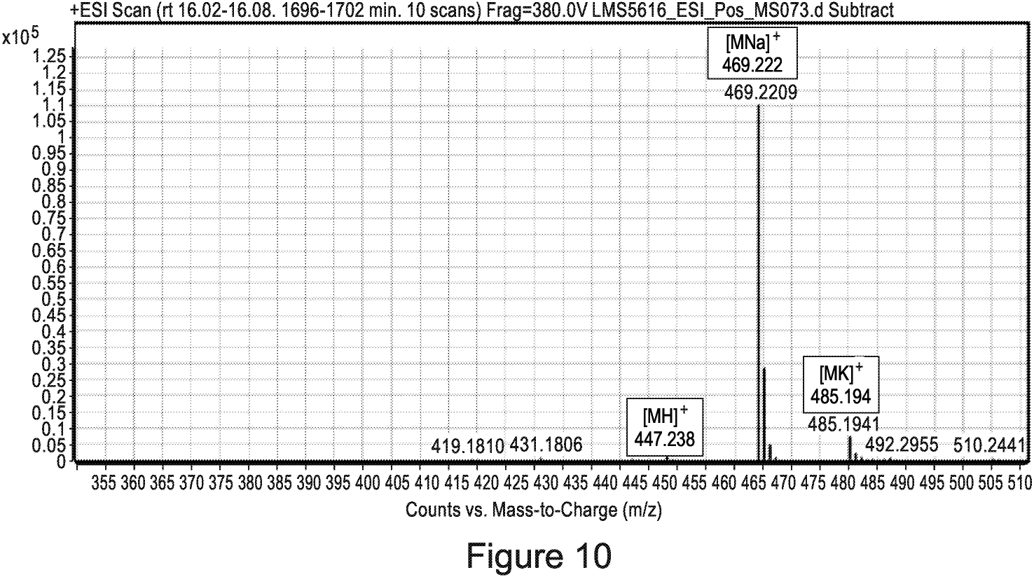

HPLC High Resolution MS (QTOF) analysis was carried out using ESI (positive mode) in order to determine Exact (Monoisotopic) mass and Empiric formula of active compound. The MS spectrum of isolated compound provides the follow information:
Molecular Formula: C₂₅H₃₄O₇
Monoisotopic Mass: 446.2304 Da
[M+H]⁺: 447.2377 Da
(FIG. 9 and FIG. 10).

### The Elucidation of the Isolated Compound by NMR Techniques.

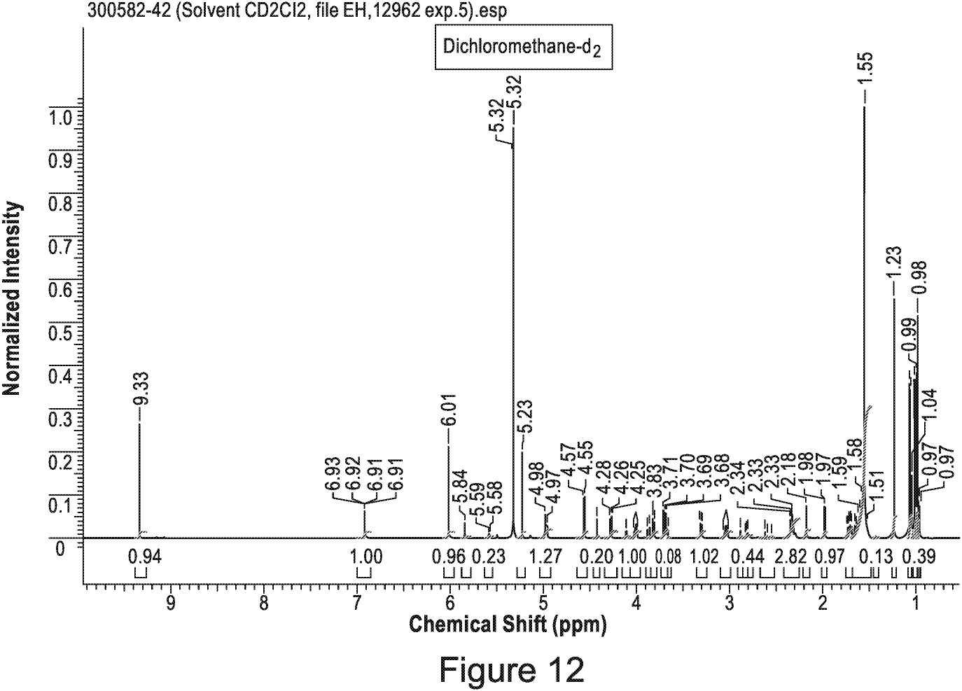

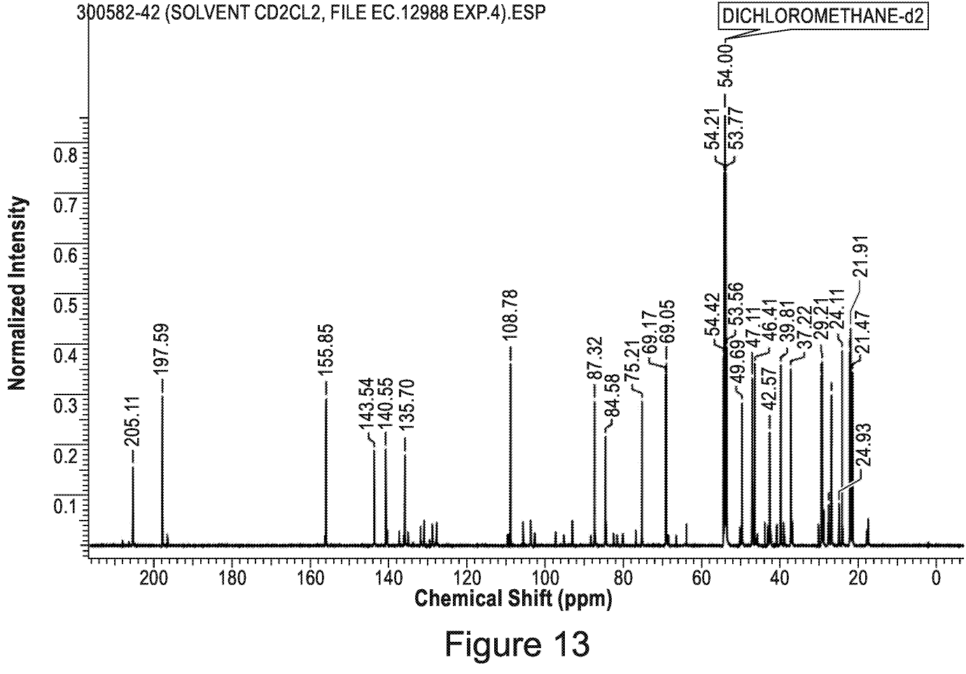

The isolated compound “300582-42” was analyzed by ¹H-NMR (FIG. 12), ¹³C-NMR (FIG. 13), COSY, C—H correlation, DEPT, HSQC and HMBC. The spectra were obtained in d-Dichloromethane, d-Acetonitrile, d-Chloroform and d-DMSO. The best results achieved in Dichloromethane solvent. Proposed assignment of NMR signals summarized in tables 1 and 2. Interpretation of NMR data led us to the following conclusions regarding the molecular structure of isolated compound (FIG. 11).

TABLE 1

¹H-NMR interpretation of isolated compound: “300582-42”

| | | | |
|---|---|---|---|
| Chemical shifts (ppm) | Functional group | Multiplicity J(H₂) | Structure |
| 9.33 | HC═O | - | —C(9)—COH(24) |
| (singlet) | | | |
| 6.92 | HC═C | J_{8,10}=3.0 | —CH(7)—CH(8)═C(9)— |
| (dd) | | J_{8,7}=7.5 | |
| 6.01 | OH | - | —C(11)—OH(26) |
| (broad singlet) | | | |
| 5.23 | CH | - | —O—CH(2)—O— |
| (singlet) | | | |
| 4.95 | CH | J_{7,8}=7.5 | —CH(8)—CH(7)—OH(23) |
| (doublet) | | | |
| 4.56 | CH | J_{4,10}=10.1 | —CH(10)—CH(4)—O |
| (doublet) | | | |
| 4.27 | CH₂ | J_{14a,14b}=11.8 | HO—CH(13)—CH2(14a)—O— |
| (dd) | Only one proton | J_{14a,13}=4.1 | |
| 4.00 | CH | J_{13,14a,b}=4.5 | —O—CH2(14a,b)—CH(13)—OH |
| (triplet) | | | |
| 3.2 | OH | - | —CH(13)—OH(28) |
| (broad doublet) | | | |
| 3.7 | CH₂ | J_{14b,14a}=11.8 | HO—CH(13)—CH₂(14b)—O— |
| (dd) | Only one proton | J_{14b,13}=5.1 | |
| 3.31 | CH | J_{10,4}=10.1 | —C(9)—CH(10)—C(11)— |
| (dd) | | J_{10,8}=2.8 | |
| 3.04 | CH | J_{30,31+32}=6.75 | —C(20)—CH(30)—(CH₃)₂(31,32)— |
| (Septet) | | | |
| 2.35 | CH₂ | - | —C(20)—CH₂(19)—CH₂(18)— |
| (multiplet) | | | |
| 2.18 | CH | - | —CH(7)—CH(6)—C(5)— |
| (broad singlet) | | | |
| 1.98 | OH | - | —CH(7)—OH(23) |
| (doublet) | | | |
| 1.71 | CH₂ | - | CH₃—C(17)—CH₂(16a)—CH₂(15)— |
| (multiplet) | Only one proton | | |
| 1.61 | CH₂ | - | CH3—C(5)—CH2(15)—CH₂(16)—CH3—C(17)—CH₂(18)—CH₂(19)— |
| (multiplet) | | | |
| 1.55 | CH₂ | - | CH₃—C(17)—CH₂(16b)—CH₂(15) |
| (multiplet) | Only one proton | | |
| 1.23 | CH₂ | - | CH₃(22)—C(5)— |
| (singlet) | | | |
| 1.06 | CH₂ | J_{31,32}=6.7 | CH₃(31/32)—CH(30)— |
| (doublet) | | | |
| 1.00 | CH₂ | J_{31,32}=6.8 | CH₃(31/32)—CH(30)— |
| (doublet) | | | |
| 0.98 | CH₂ | - | CH₃(29)—C(17)— |
| (singlet) | | | |

* Only peaks that belong to the main component are noted.

TABLE 2

¹³C-NMR (H decoupled) interpretation of isolated compound: “300582-42”

| Chemical shifts (ppm) | Functional group | Carbon number |
|---|---|---|
| 205.11 | C═O | C12 |
| 197.59 | HC═O | C24 |
| 155.85 | C═CH | C8 |
| 143.54 | C═CH | C9 |
| 140.55 | C═C | C20 |
| 135.70 | C═C | C21 |
| 108.78 | O—CH—O | C2 |
| 87.32 | CH—O— | C4 |
| 84.58 | C—OH | C11 |
| 75.21 | CH—OH | C13 |
| 69.17 | CH—OH | C7 |
| 69.05 | CH₂—O— | C14 |
| 49.69 | —C—CH₂— | C17 |
| 47.11 | CH—CH | C10 |
| 46.41 | CH—CH—OH | C6 |
| 42.57 | CH—C—CH₃ | C5 |
| 39.81 | CH₂—CH₂ | C16 |
| 37.22 | CH₂—CH₂ | C15 |
| 29.45 | CH₂—CH₂ | C18 |
| 29.21 | CH₂—CH₂ | C19 |
| 26.90 | CH₃—CH—CH₃ | C30 |
| 24.11 | CH₃ | C29 |
| 22.15 | CH₃—CH—CH₃ | C31/C32 |
| 21.91 | CH₃—CH—CH₃ | C31/C32 |
| 21.47 | CH₃ | C22 |

* Only peaks that belong to the main component are noted.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A fractionation method of a Cyathus striatus CBS 126585 for obtaining at least one of the pure active ingredients Striatal C, Striatal C′ and Striatal D 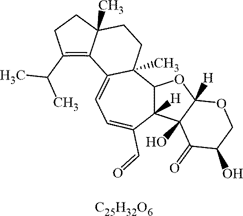 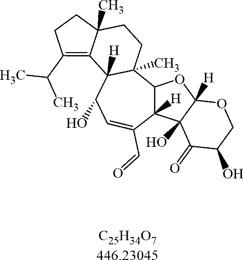 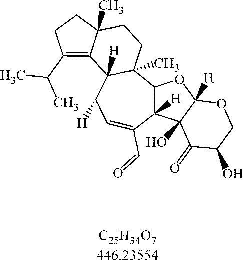
wherein the fractionation method includes:
providing an octadecyl silica gel column; and
performing a series of RP-18 preparative chromatography steps using the acetadecyl silica gel column,
wherein the elution solvents are double distilled water (DDW, solvent A) and acetonitrile (ACN, solvent B), wherein the first step includes eluting with 100% solvent A and 0% solvent B, and wherein the amount of solvent A is decreased by 5% and the amount of solvent B is increased by 5% in each 10 minute interval, until reaching a solvent comprising 100% solvent B, and wherein the elution solvents are passed through the octadecyl silica gel column at a predefined flow rate.

2. The fractionation method according to claim 1, further comprising passing 100% solvent B through the octadecyl silica gel column for an additional 20-40 minutes.

3. The fractionation method according to claim 1, wherein the predefined flow rate is 3-7 ml/min.

4. The fractionation method according to claim 1, wherein the purity of the active ingredients is between 95% to 100%.

5. A compound of the formula: 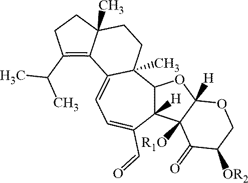
wherein R₁and R₂ may be the same or different and are selected from the group consisting of H, linear, branched or cyclic (C₁-C₆)alkyl, linear branched or cyclic (C₁-C₆)heteroalkyl, linear branched or cyclic (C₁-C₆)alkylidene, protecting group, and (C₁-C₆)aldehyde.

6-8. (canceled)

9. A composition comprising the compound according to claim 5.

10. The composition according to claim 9, further comprising a carrier, diluent, solvent, buffer, fragrance, colorants, taste enhancers, polyethylene glycol (PEG), albumin, nanoparticles, or any combination thereof.

11. The composition according to claim 9, further comprising, or for administration together with, an additional active ingredient.

12. The composition according to claim 11, wherein the additional active ingredient is an anti-cancer drug.

13. The composition according to claim 9, further comprising, or for administration together with, a food supplement, a probiotic, a prebiotic, a nutraceutical, a beverage product, a cosmetic product, or any combination thereof.

14. The composition according to claim 9, further comprising, or for administration together with, a biomass of a medicinal mushroom, an extract thereof, or any combination thereof.

15. The composition according to claim 14, wherein the medicinal mushroom is selected from the group consisting of Lentinus edodes, Coprinus, and Tremella species.

16. The composition according to claim 9, further comprising, or for administration together with, a Cyathus striatus CBS 126585 extract.

17. A method for treatment of cancer in an individual in need thereof, comprising administering to said individual a therapeutically effective amount of a compound selected from the group consisting of Striatal A, wherein R₁ and R₂ may be the same or different and are selected from the group consisting of H, linear, branched or cyclic (C₁-C₆)alkyl, linear, branched or cyclic (C₁-C₆)heteroalkyl, linear, branched or cyclic (C₁-C₆)alkylidene, protecting group, and (C₁-C₆)aldehyde; Striatal C; Striatal C′; and Striatal D 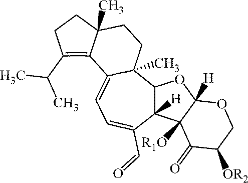 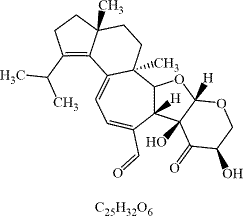 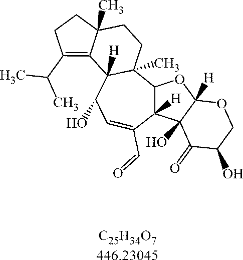 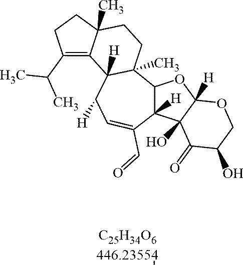
.

18. The method according to claim 17, wherein the cancer is epithelial cell cancer.

19. The method according to claim 17, wherein the cancer is pancreatic cancer, breast cancer, colon and rectal cancer, head and neck cancer, chronic myelogenous leukemia (CML), prostate cancer, ovarian cancer or uterine cancer.
